(19) United States
(12) Patent Application Publication
(10) Pub. No.: US 2011/0106027 A1
(43) Pub. Date: May 5, 2011

(54) Chemically Coated Screen for Use with Hydrophobic Filters

(75) Inventors: Mark A. Vess, Hanson, MA (US); Dinesh Aggarwal, New Delhi (IN)

(73) Assignee: TYCO HEALTHCARE GROUP LP, Mansfield, MA (US)

(21) Appl. No.: 12/893,257

(22) Filed: Sep. 29, 2010

Related U.S. Application Data
(60) Provisional application No. 61/258,253, filed on Nov. 5, 2009.

(51) Int. Cl. A61M 1/00

(52) U.S. Cl. 604/319

(57) ABSTRACT

A system for subatmospheric pressure therapy in connection with healing a surgical or chronic wound includes a wound dressing adapted for positioning relative to a wound bed of a subject and a subatmospheric pressure mechanism. The subatmospheric pressure mechanism includes a housing; a vacuum source disposed in the housing and associated with a vacuum port; a collection canister defining an internal chamber in fluid communication with the vacuum source through the vacuum port and with the wound dressing for collecting exudates removed from the wound bed under influence of the vacuum source; a hydrophobic filter in fluid communication with the vacuum source and the internal chamber of the collection canister; and a screen disposed proximally of the filter. The filter is adapted to prevent exudates from reaching the vacuum source while allowing passage of air to the vacuum source. The screen includes compounds for cleaving proteins in the exudates to prevent clogging of the filter by the exudates in the collection canister.

### CROSS-REFERENCE TO RELATED APPLICATIONS

This application claims the benefit of, and priority to, U.S. Provisional Application No. 61/258,253, filed on Nov. 5, 2009, the entire disclosure of which is incorporated by reference herein.

### BACKGROUND

1. Technical Field

The present disclosure relates to treating an open wound, and, more specifically, relates to a wound therapy system including a chemically treated screen for releasing compounds for the long term maintenance of hydrophobic filters used in negative pressure wound therapy pumps.

2. Description of Related Art

Wound closure involves the migration of epithelial and subcutaneous tissue adjacent the wound towards the center and away from the base of the wound until the wound closes. Unfortunately, closure is difficult with large wounds, chronic wounds or wounds that have become infected. In such wounds, a zone of stasis (i.e. an area in which localized swelling of tissue restricts the flow of blood to the tissues) forms near the surface of the wound. Without sufficient blood flow, the epithelial and subcutaneous tissues surrounding the wound not only receive diminished oxygen and nutrients, but, are also less able to successfully fight microbial infection and, thus, are less able to close the wound naturally. Such wounds have presented difficulties to medical personnel for many years.

Negative pressure wound therapy, also known as suction or vacuum therapy, has been used in treating and healing wounds. Application of negative pressure, e.g. reduced or subatmospheric pressure, to a localized reservoir over a wound has been found to assist in closing the wound by promoting blood flow to the area, stimulating the formation of granulation tissue, and encouraging the migration of healthy tissue over the wound. Negative pressure may also inhibit bacterial growth by drawing fluids from the wound such as exudates, which may tend to harbor bacteria. This technique has proven particularly effective for chronic or healing-resistant wounds, and is also used for other purposes such as post-operative wound care.

Generally, negative pressure therapy provides for a wound to be covered to facilitate suction at the wound area. A conduit is introduced through the wound covering to provide fluid communication to an external vacuum source. Atmospheric gas, wound exudates, or other fluids may thus be drawn from the reservoir through the fluid conduit to stimulate healing of the wound. Exudates drawn from the reservoir may be deposited in a collection canister. The canister of the wound therapy system may require disconnection or replacement for a variety of reasons, such as when filled with exudates, or if exudates escape and/or clog the filter or electronics of the system. It would be advantageous to provide a collection canister which seals the contents therein and precludes the escape of the contents when the canister is tilted or oriented with the filter side down while inhibiting clogging of the filter with exudates in the event that exudates contact the filter.

### SUMMARY

A system for subatmospheric pressure therapy in connection with healing a surgical or chronic wound includes a wound dressing adapted for positioning relative to a wound bed of a subject and a subatmospheric pressure mechanism. The subatmospheric pressure mechanism includes a housing, a vacuum source disposed in the housing and associated with a vacuum port, a collection canister defining an internal chamber in fluid communication with the vacuum source through the vacuum port and with the wound dressing for collecting exudates removed from the wound bed under influence of the vacuum source, a hydrophobic filter in fluid communication with the vacuum source and the internal chamber of the collection canister, and a screen disposed proximally of the filter. The filter is adapted to prevent exudates from reaching the vacuum source while allowing passage of air to the vacuum source. The screen includes compounds for cleaving proteins in the exudates to prevent clogging of the filter by the exudates in the collection canister.

The compounds immobilized on the screen include proteases and nucleases for breaking down proteins. The compounds may be coated on the screen by powder or crystalline coating or may be molded onto the surface of the screen. Upon contact with exudates, the compounds may activate and degrade proteins and other compounds contained therein.

### BRIEF DESCRIPTION OF THE DRAWINGS

Various embodiments of the wound dressing system of the present disclosure are described herein with reference to the drawings wherein:

FIG. 1 is a view in partial cross-section of a wound therapy system of the present disclosure illustrating the wound dressing and the subatmospheric pressure mechanism;

FIGS. 2A and 2B are cross-sectional views of alternate embodiments of the subatmospheric pressure mechanism of the wound therapy system of the present disclosure;

FIG. 3 is a perspective view of a canister insert in accordance with the principles of the present disclosure;

FIG. 4 is a schematic illustration of a filter and screen combination for use with the wound therapy system of the present disclosure; and

FIG. 5 is a schematic illustration of an alternate filter and screen combination for use with the wound therapy system of the present disclosure.

### DESCRIPTION OF THE EMBODIMENTS

The wound therapy system of the present disclosure promotes healing of a wound via the use of a wound dressing and a subatmospheric pressure mechanism. Generally, the subatmospheric pressure mechanism applies subatmospheric pressure to the wound to effectively remove wound fluids or exudates captured within the boundary of the composite wound dressing, and to increase blood flow to the wound bed and enhance cellular stimulation of epithelial and subcutaneous tissue. The wound therapy system may be entirely portable, i.e., it may be worn or carried by the subject such that the subject may be completely ambulatory during the therapy period. The wound therapy system including the subatmospheric pressure mechanism and components thereof may be entirely reusable or may be entirely disposable after a predetermined period of use or may be individually disposable whereby some of the components are reused for a subsequent therapy application.

The wound therapy system of the present disclosure promotes healing of a wound in conjunction with subatmospheric negative pressure therapy. The system may incorporate a variety of wound dressings, subatmospheric pressure sources and pumps and collection canisters. The attached figures illustrate exemplary embodiments of the present disclosure and are referenced to describe the embodiments depicted therein. Hereinafter, the disclosure will be described by explaining the figures wherein like reference numerals represent like parts throughout the several views.

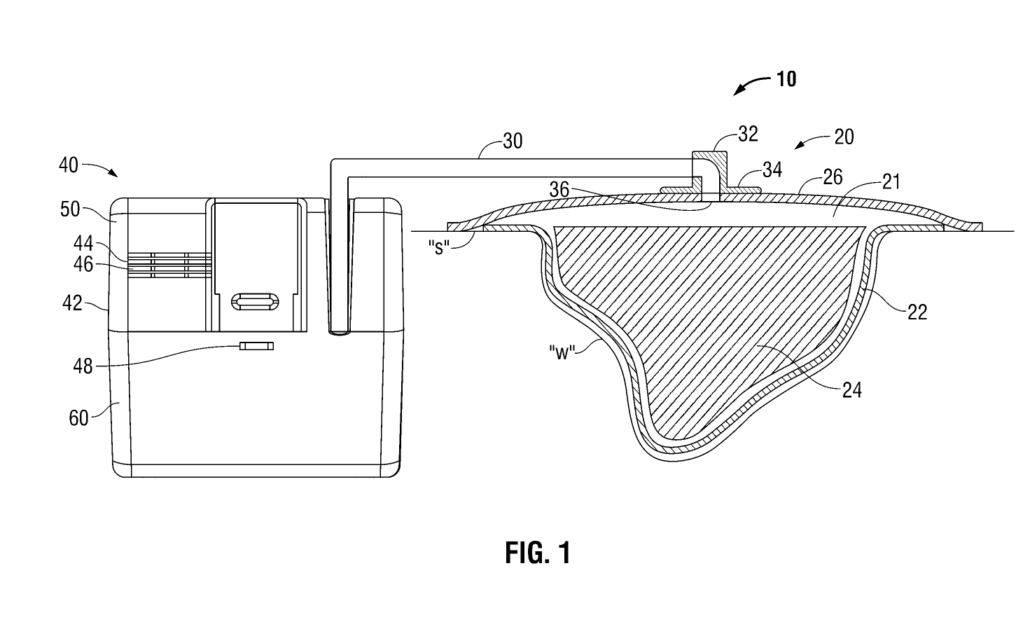

Referring initially to FIG. 1, the wound therapy system 10 according to the present disclosure is illustrated for use on a wound “w” surrounded by healthy skin “s.” Wound therapy system 10 includes composite wound dressing 20 and subatmospheric pressure mechanism 40 in fluid communication with the wound dressing 20 through conduit 30.

Wound dressing 20 is positioned relative to the wound “w” to define a reservoir 21 in which a negative pressure appropriate to stimulate healing may be maintained. Wound dressing 20 may include several components, namely, wound contact layer or member 22, a wound packing member or filler 24 supported by the contact member 22, and outer layer or cover member 26. Wound contact member 22 is adapted to substantially conform to the topography of a wound bed “w.” Wound contact member 22 may be substantially porous or perforated to permit exudates to pass from the wound bed “w” through the wound contact member 22. The passage of wound exudates through the wound contact member 22 may be unidirectional such that wound exudates do not flow back to the wound bed “w.” Unidirectional flow may be encouraged by directional apertures formed in contact member 22, lamination of materials having absorption properties differing from those of contact member 22, or by selection of materials that promote directional flow. A non-adherent material may be selected such that contact member 22 does not tend to cling to wound bed “w” or surrounding material when it is removed. Exemplary materials that may be used as a contact member 22 are sold under the trademarks XEROFORM® and CURITY®, offered by Tyco Healthcare Group LP (d/b/a Covidien).

Wound packing member 24 of wound dressing 20 is intended to transfer wound fluid and exudates. Wound packing member 24 is conformable to assume the shape of any wound bed “w” and may be packed up to the level of healthy skin “s.” Wound packing member 24 may be pre-formed in any shape and size or may be custom fit by cutting the packing member 24 to a desired shape and/or size. Wound packing member 24 may be treated with agents to promote healing of the wound, such as polyhexamethylene biguanide to decrease the incidence of infection, and other substances having clinical use, such as a dye. Suitable materials for wound packing member 24 are sold under the trademarks KERLIX®, EXCILON®, and WEBRIL®, all by Tyco Healthcare Group LP (d/b/a Covidien).

Outer member or wound covering 26 encompasses the perimeter of the wound dressing 20 to surround wound bed “w” and to provide a liquid and/or fluid tight seal around the perimeter “p” of the wound bed “w.” For instance, the sealing mechanism may be any adhesive bonded to the perimeter of wound covering 26. One exemplary material that may be used as an adhesive dressing is sold under the trademark CURAFOAM® by Tyco Healthcare Group LP (d/b/a Covidien). Thus, wound covering 26 may act as both a microbial barrier and a fluid barrier to prevent contaminants from entering wound bed “w” and for maintaining the integrity thereof.

Wound covering 26 is typically a flexible material, e.g., resilient or elastomeric, that seals the top of wound dressing 20 to prevent passage of liquids, fluids, or contamination to and from the wound dressing 20. Wound covering 26 may be formed from a moisture vapor permeable membrane to promote the exchange of oxygen moisture between the wound bed “w” and atmosphere. A membrane that provides a sufficient moisture vapor transmission rate is a transparent membrane sold under the trademark POLYSKIN® II by Tyco Healthcare Group LP (d/b/a Covidien). A transparent membrane permits an assessment of wound conditions without requiring removal of the wound covering 26. Alternatively, wound covering 26 may comprise an impermeable membrane or a substantially rigid membrane.

Wound covering 26 may include a port or connector 32 in fluid communication with the wound dressing 20 to facilitate connection of wound dressing 20 to conduit or tubing 30. Conduit 30 defines a fluid flow path leading through wound therapy system 10. Connector 32 may be configured as a rigid or flexible, low-profile component, and may be adapted to receive conduit 30 in a releasable and fluid tight manner. An adhesive on the underside of flange 34 of connector 32 may provide a mechanism for affixing the conduit 30 to the dressing or alternatively, flange 34 may be positioned within reservoir 21 such that an adhesive on an upper side of the flange 34 affixes the conduit 30. However it is affixed to wound dressing 20, a hollow interior 36 of connector 32 provides fluid communication between conduit 30 and the interior of wound dressing 20, such as reservoir 21.

Connector 32 may have a valve (not shown) built therein or in line with conduit 30, e.g., a one-way valve to permit exudates to flow in one direction only, i.e., away from wound dressing 20 toward subatmospheric pressure mechanism 40. Connector 32 may be provided as a pre-affixed component of wound dressing 20, as a component of conduit 30 or entirely separate and connected thereto by conventional means. Alternatively, connector 32 may be eliminated if other provisions are made for providing fluid communication between wound dressing 20 and conduit 30.

Conduit 30 extends from wound dressing 20 to subatmospheric pressure mechanism 40. Any suitable conduit may be used including those fabricated from flexible elastomeric or polymeric materials. Conduit 30 may connect to subatmospheric pressure mechanism 40 or other system components by conventional air tight means such as friction fit, bayonet coupling, or barbed connectors. The conduit connections may be made permanent, or alternatively a quick-disconnect or other releasable means may be used to provide some adjustment flexibility to the apparatus.

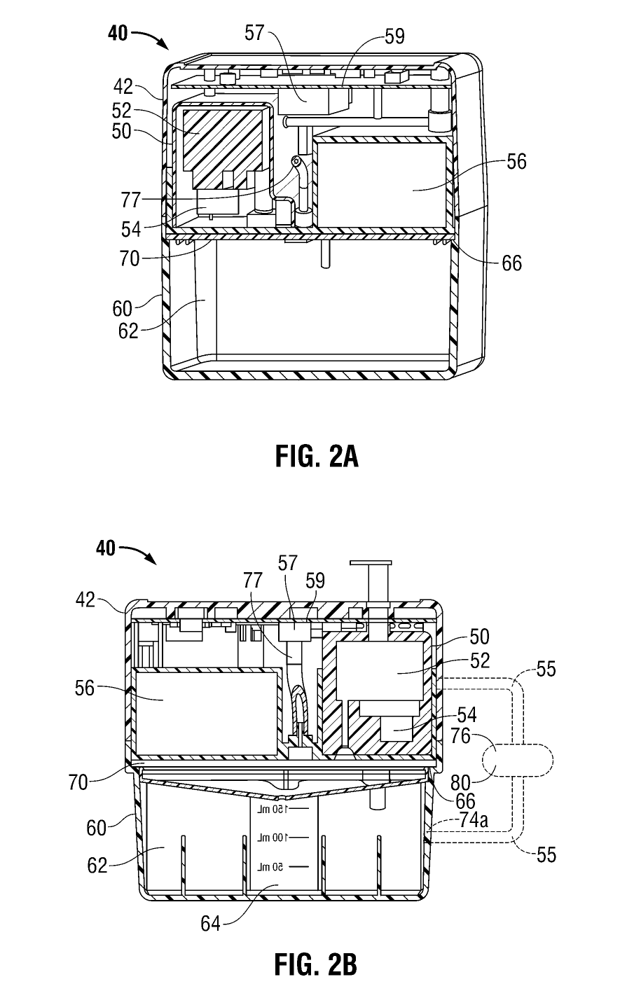

Referring now to FIGS. 2A and 2B, in conjunction with FIG. 1, subatmospheric pressure mechanism 40 will be discussed. Subatmospheric pressure mechanism 40 includes housing 42, control unit 50 disposed within the housing 42, and collection canister 60. Housing 42 may be any suitable rigid member adapted for donning by the subject. Control unit 50 may incorporate vacuum source or pump 52, actuator or motor 54 for activating the vacuum source 52, and power source 56. Vacuum source or pump 52 generates or otherwise provides negative pressure to wound therapy system 10. Vacuum source or pump 52 may be a pump of the diaphragmatic, peristaltic or bellows type or the like, in which the moving part(s) draw exudates out of the wound bed “w” into the wound dressing 20 by creating areas or zones of decreased pressure e.g., vacuum zones with the wound dressing 20 appropriate to stimulate healing of the wound. This area of decreased pressure may communicate with the wound bed “w” to facilitate removal of the fluids therefrom and into packing member 24.

Vacuum source or pump 52 may be a miniature pump or micropump that may be biocompatible and adapted to maintain or draw adequate and therapeutic vacuum levels. The vacuum level of subatmospheric pressure achieved may be in the range of about 20 mmHg to about 500 mmHg. In embodiments, the vacuum level may be about 75 mmHg and about 125 mmHg, or between about 30 mmHg and 80 mmHg. Vacuum source or pump 52 is actuated by actuator 54 which may be any means known by those skilled in the art, including, for example, AC motors, DC motors, voice coil actuators, solenoids, etc. In embodiments, actuator 54 may be incorporated within pump 52.

Power source 56 may be disposed within housing 42 or separately mountable to the housing 42. A suitable power source 56 includes alkaline batteries, wet cell batteries, dry cell batteries, nickel cadmium batteries, solar generated means, lithium batteries, NiMH batteries (nickel metal hydride) each of which may be of the disposable or rechargeable variety.

Subatmospheric pressure mechanism 40 may also include a pressure transducer 57 which may be attached to a printed circuit board (PCB) 59. Within the PCB 59 is software or circuitry that powers the pressure transducer 57 and receives its pressure signals (i.e., electrical signals indicative of the negative pressure being measured).

Housing 42 may further include vent portal 44 configured to vent exhaust air from vacuum source or pump 52 through an exhaust port (not shown). Vent portal 44 extends from housing 42 and may be directly connected to vacuum source 52. It is also envisioned that vent portal 44 may exhaust air from within housing 42 rather than directly from vacuum source 52. Vent portal 44 may include filter 46 extending across the vent portal 44. Filter 46 may be a bacterial filter including charcoal or other odor absorbing materials to help prevent emission of bacteria from housing 42.

Collection canister 60 collects exudates removed from the wound bed “w” during therapy through conduit or tubing 30. Collection canister 60 is associated with housing 42 and may be incorporated within the housing 42 or releasably connected to the housing 42 by conventional means. Housing 42 and collection canister 60 of subatmospheric pressure mechanism 40 may be releasably coupled via mating members 48. Mechanisms for selective coupling and decoupling of housing 42 and collection canister 60 include fasteners, latches, clips, straps, bayonet mounts, magnetic couplings, and other devices for selective mating of housing 42 and collection canister 60.

Collection canister 60 may comprise any container suitable for containing wound fluids and is substantially rigid defining an internal chamber 62 in fluid communication with tubing 30. In the alternative, collection canister 60 may be relatively flexible. Collection canister 60 may contain an absorbent material to consolidate or contain the wound drainage or debris, such as silica gel. In embodiments, at least a portion of collection canister 60 may be transparent to assist in evaluating the color, quality, or quantity of wound exudates. A transparent portion or window 64 may thus assist in determining the remaining capacity of the canister 60 or when the canister 60 should be replaced.

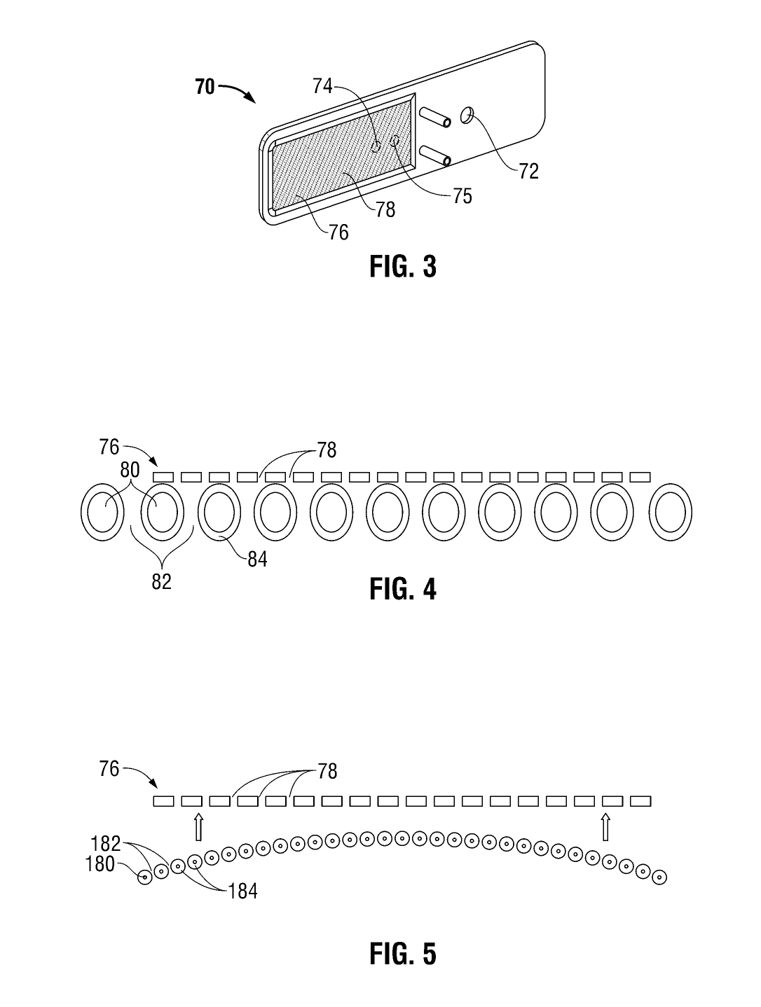

Collection canister 60 may include a canister insert 70. Referring now to FIG. 3, in conjunction with FIGS. 2A and 2B, canister insert 70 is dimensioned to fill the opening of canister 60 and be placed within internal chamber 62 of canister 60 until it engages a lip 66 around at least a portion of the peripheral inner edge of canister 60 or frictionally engages the inner walls of the canister in a fluid tight, yet releasable manner. Canister insert 70 include fluid inlet 72, suction port 74 (shown in phantom), and a pressure transducer port 75 (shown in phantom). The suction port 74 and the pressure transducer port 75 are disposed beneath filter 76. Fluid inlet 72 depends from a planar segment of canister insert 70 and is configured to operably engage conduit 30. Fluid inlet 72 may be connectable with conduit 30 by conventional air and fluid tight means, such as those described above, and terminates within internal chamber 62 to deposit exudates conveyed by the conduit 30 into the internal chamber 62. In embodiments, fluid inlet 72 may contain a luer lock or other connector within the purview of those skilled in the art to secure the end of conduit 30 with the fluid inlet 72. It is envisioned that fluid inlet 72 is configured to receive a cap in order to prevent leakage of exudates and odor from internal chamber 62 of collection canister 60 when housing 42 is separated from the canister 60.

Suction port 74 is in fluid communication with vacuum source or pump 52 and may be an opening defined in canister insert 70. Pump 52 creates a vacuum within internal chamber 62 of collection canister 60 by drawing air through suction port 74. Pressure transducer port 75 is in fluid communication with pressure transducer 57 through tube 77 and permits the monitoring of pressure levels within internal chamber 62 of collection canister 60.

Referring now to FIG. 4, canister insert 70 includes a filter 76, such as a hydrophobic membrane or baffling, including pores 78 to prevent exudates from being aspirated into pump 52. Filter 76 is attached to canister insert 70 through conventional means, such as mechanical binding. The filter 76 may be dimensioned to span the lower surface of canister insert 70 to cover suction port 76 and pressure transducer port 75. The filter 76 is disposed adjacent to or within suction port 74 such that suction port 74 passes air between vacuum source 52 and the canister 60 through filter 76 while keeping the contents of the canister from reaching the vacuum pump 52 or other components of control unit 50. The filter 76 also prevents migration of the fluids or exudates into the pressure transducer port 75 and pressure transducer 57.

The hydrophobic nature of the filter 76 allows the canister 60 to be oriented in a way other than with the pump 52 above the canister 60, such as on the side of the canister 60 or tipped, without exudates in the canister 60 being aspirated into the pump 52. Some portion of the surface of the filter 76 remains uncovered, thereby allowing continued flow of air to vacuum pump 52.

The pores 78 of the filter 76, however, may become clogged over time as a result of exudates coming in contact with the filter's surface. Protein strands and other exudates compounds may become lodged in the pores 78 of the filter 76, thereby reducing air flow through filter 76. A chemically treated screen 80 may be disposed adjacent to the surface of the filter 76 facing the canister 60 to break down the proteins and other compounds in the exudates, and proteins thereof trapped in the filter's pores 78, to preserve the ability of the screen 80 to function over time. Cleavage of large compounds in the exudates, such as proteins, nucleic acids, lipids, and polysaccharides, into smaller units may prevent these compounds from becoming trapped in the filter's pores 78.

The screen 80 may comprise a fine mesh of plastic or other inert material. Exemplary materials include, but are not limited to, polyolefins (such as polyethylene and polypropylene); polyesters (such as polyethylene terephthalate and polybutylene terephthalate); acrylic polymers and copolymers; vinyl halide polymers and copolymers (such as polyvinyl chloride); polyamides (such as nylon 4, nylon 6, nylon 6.6, nylon 610, nylon 11, nylon 12 and polycaprolactam); polyurethanes, silicones, rayon, and spandex.

The screen 80 includes openings 82 that measure about 0.01 inches to about 0.03 inches across. In embodiments, the size of the openings 82 are 0.02 inches.

The screen 80 also contains compounds 84 which are immobilized to the surface of the screen 80. Compounds 84 include various proteases, nucleases, and proteins which have the ability to cleave peptide bonds thus reducing the molecular weight of a protein strand and creating smaller sized peptides. By reducing the size of the proteins in the exudates, clogging of the hydrophobic filter 76 may be eliminated or at least diminished so that the filter 76 remains functional. Examples of proteases include papain, trypsin, cathepsin, plasmin, pepsin, chymotrypsin, thermolysin, carboxypeptidase Y, Glu-C, Asp-N, Lys-C, and combinations thereof, such as Glu-C/Trypsin, Glu-C/Chymotrypsin, and Trypsin/Asp-N, for cleavage at different sites along the protein. A variety of nucleases, including exo- and/or endo-nucleases, may be used with screen 80 as is within the purview of those skilled in the art. Nucleases include a variety of restriction enzymes, DNA, and RNA which can catalyze various reactions, such as the cleavage of DNA, hydrolysis of RNA, and combinations thereof. Moreover, other bioactive agents may be combined with the compounds 84 or coated on the screen 80, such as anti-adhesives, antimicrobials, anti-infectives, anti-thrombotics, and other substances which may aid in maintaining a clean filter as is within the purview of those skilled in the art.

The compounds 84 are coated, bonded, or otherwise applied to screen 80 by any method within the purview of those skilled in the art. Exemplary methods include, for example, powder coating, crystalline coating, and molding. Powder coating may include dry coatings of compound 84 which do not require a solvent. Crystalline coatings may include a solution of compound 84 and solvent which may be deposited on the screen 80 via dipping, spinning, brushing, spraying, and other means within the purview of those skilled in the art. Through the use of a polymerization, condensation, or heating process, the compounds 84 form a crystalline coating.

The screen 80 may be adapted and configured to conform to the surface of the filter 76 to ensure contact with the filter 76 over its entire surface. The screen 80 may be placed in close proximity to the filter 76, and in embodiments, may be mechanically bonded to filter 76. The screen 80 may cover substantially the entire filter 76 or, in embodiments, may cover a majority of the filter 76.

In embodiments, screen 180 is fabricated with a slight curvature over its surface as illustrated in FIG. 5. This curvature allows the screen 180 to flatten out onto the filter 76 upon movement of exudates in the direction of the arrows to ensure contact over a majority of the filter's surface. This configuration places the compounds 184 of screen 180 in contact with or in close proximity to a large number of pores 78 of filter 76. As exudates come in contact with the screen 180 and filter 76, some of the compounds 184 of screen 180 may dissolve, or otherwise become active, and remain in the pores 78 of the filter 76. Although the compounds 184 may not reach all of the pores 78, enough of the pores 78 remain open so as to preserve the functionality of the filter 76 over time and exposure to exudates. The compounds 184 may also attach to any protein deposits that may come in contact with and/or adhere to the surface of the screen 180. As the exudates are sloshed around the canister 60, the movement of the fluid may allow any accumulated debris to be washed away from the screen 180. In embodiments, the curvature of the screen 180 may be concave or convex, and likewise, the filter 76 may also possess a slight convex or concave curvature independent of, or complimentary to, the curvature of the screen 180.

Alternatively, the filter 76 and screen 80 combination may be disposed in another intermediary location between pump 52 and internal chamber 62 of canister 60, e.g., with the filter 76 and the screen 80 external of internal chamber 62. As illustrated in phantom in FIG. 2B, filter 76 and screen 80 may be disposed along tube 55 which connects suction port 74A of canister 60 with vacuum pump 52.

In an exemplary embodiment of use, the wound dressing 20 is placed adjacent the wound bed “w” and connected to subatmospheric pressure mechanism 40 via tubing 30, as illustrated in FIG. 1. Housing 42 and canister 60 are connected if not already connected to each other. Control unit 50 of subatmospheric pressure mechanism 40 is then activated creating a reduced pressure state within wound dressing 20. Vacuum source or pump 52 may be set at a specific set point whereby the pump will begin to draw vacuum until it achieves the set point as detected, e.g., by a pressure transducer. As the pumping progresses, exudates are collected and directed to collection canister 60. The vacuum reading at the pump 52 will stay at this level until the set point is changed, the pump is turned off, or there is a major leak in the system that overcomes the pump's ability to continue to achieve this level. Subatmospheric pressure therapy may be continuous or intermittent.

In the event that exudates contact filter 76, such as by tilting or inversion of the canister 60, the screen 80 provides protection to the filter 76 by dissolving and/or degrading proteins and compounds in the exudates that may accumulate on the surface of the screen 80 and/or filter 76. The screen 80 effectively forms an active protective barrier over the hydrophobic filter 76.

While the disclosure has been illustrated and described, it is not intended to be limited to the details shown, since various modifications and substitutions can be made without departing in any way from the spirit of the present disclosure. As such, further modifications and equivalents of the disclosure herein can occur to persons skilled in the art, and all such modifications and equivalents are believed to be within the spirit and scope of the disclosure as defined by the following claims.

What is claimed is:

1. A system to promote the healing of an exuding wound, which comprises:
a wound dressing dimensioned for positioning relative to a wound bed of a subject; and
a subatmospheric pressure mechanism including:
a housing;
a vacuum source disposed in the housing and associated with a vacuum port;
a collection canister defining an internal chamber in fluid communication with the vacuum source through the vacuum port and with the wound dressing for collecting exudates removed from the wound bed under influence of the vacuum source;
a hydrophobic filter in fluid communication with the vacuum source and the internal chamber of the collection canister, the filter adapted to prevent exudates from reaching the vacuum source while allowing passage of air to the vacuum source; and
a screen including compounds for cleaving proteins in the exudates, the screen disposed proximally of the filter to prevent clogging of the filter by the exudates in the collection canister.

2. The system according to claim 1, wherein the compounds are proteases.

3. The system according to claim 2, wherein the proteases are one of papain, trypsin, cathepsin, thermolysin, plasmin, pepsin, chymotrypsin, carboxypeptidase Y, Glu-C, Asp-N, Lys-C, and combinations thereof.

4. The system according to claim 1, wherein the compounds are nucleases.

5. The system according to claim 4, wherein the nucleases are one of DNA, RNA, and combinations thereof.

6. The system according to claim 4, wherein the nucleases are exo- and/or endo-nucleases.

7. The system according to claim 1, wherein the screen is mechanically bonded to the filter.

8. The system according to claim 1, wherein the screen is disposed adjacent to the filter.

9. The system according to claim 1, wherein the filter and the screen are disposed in a canister inlet.

10. The system according to claim 9, wherein the filter is disposed substantially planar to the canister inlet.

11. The system according to claim 10, wherein the screen is disposed at a slight curvature relative to the filter.

12. The system according to claim 1, wherein the compounds are immobilized on the screen via one of powder coating, crystalline coating, and molding.
